(19) United States
(12) Patent Application Publication
(10) Pub. No.: US 2026/0004900 A1
(43) Pub. Date: Jan. 1, 2026

(54) SYSTEMS AND METHODS FOR COMPREHENSIVE PATIENT MONITORING AND VALIDATED ELECTRONIC-MEDICAL-RECORDS REPORTING FOR HEALTHCARE FACILITIES

(71) Applicant: I.AiOmed Technologies LTD, Tel-Aviv (IL)

(72) Inventors: Keren LIPSHITZ, Tel-Aviv (IL); Leon REMENNIK, Haifa (IL)

(21) Appl. No.: 18/758,758

(22) Filed: Jun. 28, 2024

(51) Int. Cl. G16H 10/60; G16H 15/00

(52) U.S. Cl. CPC G16H 10/60; G16H 15/00

(57) ABSTRACT

A system and a method for validated Electronic-Medical-Record (EMR) reporting, configured to access a set of measurements of one or more health indicators for one or more patients including values, identify invalid values and abnormal values of measurements in the set of measurements and indicate the identified invalid values and abnormal values of measurements. The system and method are further configured to report the set of measurements to an EMR system, where each measurement having an invalid value is reported to the EMR system only after a valid value is received for the measurement and the invalid value is replaced by the valid value, and each measurement having an abnormal value is reported to the EMR system only after the abnormal value is approved, or a normal value is received for the measurement and the abnormal value is replaced by the normal value.

### FIELD

The present disclosure relates generally to patient monitoring, and more specifically, to systems and methods for patient monitoring and validated Electronic-Medical-Records (EMR) reporting for healthcare facilities such as long-term and sub-acute care facilities.

### BACKGROUND

Long-term care facilities are generally for patients who have temporary or long-term disabilities that interfere with mobility or their ability to manage basic activities of daily living, such as bathing, dressing or toileting, is affected. Sub-acute care facilities and Skilled Nursing Facilities (SNFs) are generally for those patients who are critically ill or suffer an injury, do not require hospital acute care, but do require more intensive licensed skilled nursing care than is provided to the majority of patients in a skilled nursing facility.

Patients’ health indicators, such as vital signs or any other medical or physiological data that is required according to the patient’s treatment plan may be monitored and measured manually, semi-automatically or automatically, continuously, and non-continuously, and may be recorded in electronic medical records (EMRs), e.g., of a long-term care facility, a sub-acute care facility or an SNF.

Monitoring patient’s health indicators, such as vital signs, e.g., blood pressure, blood oxygen saturation, body temperature, blood oxygen saturation, heart rate, respiration rate, electrocardiography (ECG), glucose levels, activity level, the amount of urine and the like may be performed by a plurality of technologies provided by different manufacturers. The different monitoring devices may be stationary or mobile, such as wearable devices.

### SUMMARY

The present disclosure relates to systems and methods for patients’ monitoring in healthcare facilities such as long-term and sub-acute care facilities. More particularly, the present disclosure relates to systems and methods for patient monitoring and validated EMR reporting.

In accordance with aspects of the present disclosure, a system for validated Electronic-Medical-Record (EMR) reporting includes at least one hardware processor and at least one computer readable storage device storing instructions for execution by the at least one hardware processor, the instructions, when executed, cause the system to access a set of measurements of one or more health indicators for one or more patients, where the set of measurements includes values of measurements of the set of measurements, identify invalid values and abnormal values of measurements in the set of measurements, indicate the identified invalid values and abnormal values of measurements, and report the set of measurements to an EMR system. The reporting to the EMR system includes, for each measurement having an invalid value, reporting the measurement to the EMR system only after a valid value is received for the measurement and the invalid value is replaced by the valid value, and for each measurement having an abnormal value, reporting the measurement to the EMR system only after the abnormal value is approved or a normal value is received for the measurement and the abnormal value is replaced by the normal value.

In various embodiments of the system, the system further includes a communication device configured to receive measurement data from a plurality of medical measurement devices, where the instructions, when executed, further cause the system to store the received measurement data in the at least one computer readable storage device.

In various embodiments of the system, indicating the identified invalid values of measurements and the identified abnormal values of measurements includes displaying, on a display device, the set of measurements where the identified invalid values and the identified abnormal values are visually indicated.

In various embodiments of the system, the instructions, when executed, further cause the system, for each measurement having an invalid value, to request a user to repeat the respective measurement, or to allow the user to edit the invalid value.

In various embodiments of the system, if the user has an adequate permission, the user is allowed to edit the invalid value, and if the user does not have an adequate permission, the user is requested to repeat the respective measurement.

In various embodiments of the system, the instructions, when executed, further cause the system to allow the approval of an abnormal value of a measurement of the set of measurements only by a user having an adequate permission.

In various embodiments of the system, the instructions, when executed, further cause the system to allow editing an abnormal value of a measurement of the set of measurements only by a user having an adequate permission.

In various embodiments of the system, identifying invalid values of measurements in the set of measurements includes determining a value of a measurement of the set of measurements as invalid if the value is not within a predefined range.

In various embodiments of the system, the identifying of the abnormal values of measurements in the set of measurements includes evaluating each value of the values of the measurements based on the medical condition of a patient from whom the respective measurement was taken.

In various embodiments of the system, each patient is associated with a predefined profile of a set of predefined profiles, each profile of the set of predefined profiles includes one or more ranges of values of one or more measurements of health indicators, and each range of values of the one or more ranges of values is defined for a different type of measurement.

In various embodiments of the system, evaluating each value of the values of the measurements of the set of measurements includes determining a value of a measurement of the set of measurements as abnormal if the value of the respective measurement is not within the range of values defined for the respective measurement in the predefined profile of the set of predefined profiles associated with the patient from whom the respective measurement was taken.

In various embodiments of the system, the instructions, when executed, further cause the system to allow editing the predefined set of profiles.

In various embodiments of the system, the reporting of the set of measurements to the EMR system is performed in a batch.

In various embodiments of the system, the reporting of the set of measurements to the EMR system is performed individually.

In various embodiments of the system, The system the set of measurements of one or more health indicators is continuously updated and the reporting to the EMR system is performed according to one or more predefined rules.

In various embodiments of the system, the reporting to the EMR system is performed at predefined times.

In various embodiments of the system, the reporting further includes, for each measurement of the set of measurements missing a value and of a type defined as an essential type of measurement, reporting the respective measurement to the EMR system only after the lack of value is approved or a value is received for the respective measurement.

In various embodiments of the system, the approval of lack of value may be performed only by a user having an adequate permission.

In various embodiments of the system, the instructions, when executed, further cause the system to receive the value for the respective measurement by allowing a user who has an adequate permission to enter the value.

In various embodiments of the system, the instructions, when executed, further cause the system to store in the storage device the values of the measurements of the set of measurements except for values which are invalid.

In various embodiments of the system, the health indicators include vital signs.

In accordance with aspects of the present disclosure, a computer-implemented method for validated EMR reporting, includes accessing a set of measurements of one or more health indicators for one or more patients, where the set of measurements includes values of measurements of the set of measurements, identifying invalid values of measurements and abnormal values of measurements in the set of measurements, indicating to a user the identified invalid values of measurements, or the identified abnormal values of measurements or both, and reporting the set of measurements to an EMR system. The reporting of the set of measurements to an EMR system includes for each measurement having an invalid value, reporting the measurement to the EMR system only after a valid value is received for the measurement and the invalid value is replaced by the valid value, and for each measurement having an abnormal value, reporting the measurement to the EMR system only after the abnormal value is approved or a normal value is received for the measurement and the abnormal value is replaced by the normal value.

In various embodiments of the method, the method further includes storing the set of measurements in a storage device.

In various embodiments of the method, indicating the identified invalid values of measurements or the identified abnormal values of measurements, or both, includes displaying, on a display device, the set of measurements wherein the identified invalid values or the identified abnormal values, or both, are visually indicated.

In various embodiments of the method, the method further includes requesting a user to repeat the respective measurement or allowing the user to edit the invalid value.

In various embodiments of the method, if the user has an adequate permission, the user is allowed to edit the invalid value, and if the user does not have an adequate permission, the user is requested to repeat the respective measurement.

In various embodiments of the method, the method further includes allowing the approval of an abnormal value of a measurement of the set of measurements only by a user having an adequate permission.

In various embodiments of the method, the method further includes allowing editing an abnormal value of a measurement of the set of measurements only by a user having an adequate permission.

In various embodiments of the method, identifying invalid values of measurements in the set of measurements includes determining a value of a measurement of the set of measurements as invalid if the value is not within a predefined range.

In various embodiments of the method, the identifying of the abnormal values of measurements in the set of measurements includes evaluating each value of the values of the measurements of the set of measurements based on the medical condition of a patient from whom the respective measurement was taken.

In various embodiments of the method, each patient is associated with a predefined profile of a set of predefined profiles, each profile of the set of predefined profiles includes one or more ranges of values of one or more measurements of health indicators, and each range of values of the one or more ranges of values is defined for a different type of measurement.

In various embodiments of the method, evaluating each value of the values of the measurements of the set of measurements includes determining a value of a measurement of the set of measurements as abnormal if the value of the respective measurement is not within the range of values defined for the respective measurement in the predefined profile of the set of predefined profiles associated with the patient from whom the respective measurement was taken.

In various embodiments of the method, the method further includes allowing editing of the predefined set of profiles.

In various embodiments of the method, the reporting of the set of measurements to the EMR system is performed in a batch.

In various embodiments of the method, the reporting of the set of measurements to the EMR system is performed individually.

In various embodiments of the method, the set of measurements of one or more health indicators is continuously updated and the reporting to the EMR system is performed according to one or more predefined rules.

In various embodiments of the method, the reporting to the EMR system is performed at predefined times.

In various embodiments of the method, the reporting further includes, for each measurement of the set of measurements missing a value and of a type defined as an essential type of measurement, reporting the respective measurement to the EMR system only after the lack of value is approved or a value is received for the respective measurement.

In various embodiments of the method, the approval of lack of value may be performed only by a user having an adequate permission.

In various embodiments of the method, the method further includes receiving the value for the respective measurement by allowing a user having an adequate permission to enter the value.

In various embodiments of the method, the method further includes storing, in a storage device, the values of the measurements of the set of measurements except for values which are invalid.

In various embodiments of the method, the health indicators include vital signs.

### BRIEF DESCRIPTION OF THE DRAWINGS

The above and other aspects and features of the disclosure will become more apparent in view of the following detailed description when taken in conjunction with the accompanying drawings wherein like reference numerals identify similar or identical elements.

FIG. 1 is a diagram of a system for patient monitoring and validated EMR reporting for a healthcare facility, in accordance with aspects of the disclosure;

FIG. 2 is a flow diagram of a method for validated reporting to an EMR system, in accordance with aspects of the disclosure;

FIG. 3 is an illustration of a page of a Graphical User Interface (GUI) presenting a list of patients in a health-care facility, in accordance with aspects of the present disclosure;

FIG. 4 is an illustration of a page of the GUI of FIG. 3 for editing thresholds of a profile assigned to a patient of the healthcare facility, in accordance with aspects of the present disclosure;

FIG. 5 is an illustration of a page of the GUI of FIG. 3 presenting the status of medical devices assigned to patients of the healthcare facility and in communication with the system of FIG. 1, in accordance with aspects of the present disclosure;

FIG. 6A is an illustration of a page of the GUI of FIG. 3 presenting a list of values of measurements of vital signs to be reported to an EMR system, in accordance with aspects of the present disclosure;

FIG. 6B is an illustration of a page of the GUI of FIG. 3 presenting a status report following an EMR reporting action, in accordance with aspects of the present disclosure;

FIG. 6C is an illustration of a page of a GUI designed for a mobile computing device presenting values of measurements of vital signs for on-spot handling, in accordance with aspects of the present disclosure; and

FIG. 7 a flow diagram of an exemplary procedure for validated reporting to an EMR system, in accordance with aspects of the disclosure.

It will be appreciated that for simplicity and clarity of illustration, elements shown in the figures have not necessarily been drawn to scale. For example, the dimensions and/or aspect ratio of some of the elements can be exaggerated relative to other elements for clarity. Further, where considered appropriate, reference numerals can be repeated among the figures to indicate corresponding or analogous elements throughout the serial views.

### DETAILED DESCRIPTION

Healthcare delivery is shifting towards digital solutions to solve common challenges. EMRs, process management, and wearable devices for continuous remote patient monitoring have the potential to raise the level of care availability and quality. A comprehensive solution designed for healthcare facilities such as long-term and sub-acute care facilities, e.g., skilled nursing facilities, is desired. At least some of these technologies exist as individual siloed, and most continuous monitoring solutions are designed for patients’ use at home and are misfits for the setup of healthcare facilities such as long-term and sub-acute care facilities. Combining several systems comes at a high financial cost and operational overhead. The complexity involved has left such healthcare facilities wary of adopting new solutions, which don’t directly fit their needs and come with a steep learning curve and high operational costs.

The disclosed systems and methods provide a comprehensive, automatic or semi-automatic and adaptable (e.g., may be configured for the needs of the specific healthcare facility) solution for healthcare facilities, and for long-term and sub-acute care facilities, in particular. The disclosed systems and methods provide a comprehensive process for health indicators processing which acts as a pipeline starting with data collection, continuing to data validation, and ending with data consumption and reporting, e.g., to an EMR system. The delivery process may include a human decision support mechanism for validation of quality of health indicators values. The health indicators may be collected from various third-party medical devices of various manufacturers utilizing various monitoring or measurement technologies.

Detecting abnormalities (including marking of abnormal vitals), e.g., based on patient’s profile may be provided. For every patient the disclosed systems and methods may allocate thresholds for all the parameters that are being monitored. For example, thresholds for Heart Rate (HR), Respiratory Rate (RR), Blood Pressure (BP), body temperature etc. may be determined. The thresholds may define the maximum value and minimum value for a parameter. If a measured value exceeds the threshold, e.g., determined abnormal, the disclosed systems and methods may provide a notification to the user.

The threshold values may be defined based on the patient's clinical condition. For example, blood pressure values may be considered normal for a patient suffering from Hypertension, while for a patient who is not suffering from Hypertension, those values will be considered abnormal.

According to some aspects, for each parameter, a range of valid values may be determined. For example, Oxygen level cannot be above 100%, body temperature cannot be above 45 degrees and so on. If the parameter value is not within its associated reasonable range, the user may not be allowed to save it. Such a validated EMR reporting mechanism is particularly important in cases where the user may edit values according to the disclosed systems and methods.

Moreover, the disclosed systems and methods provide a validated EMR reporting of medical measurements. EMR reporting may include incorrect, inaccurate or missing measurements, e.g., due to human errors such as typos, which may affect the patients’ treatment or have legal consequences. The disclosed automatic validation significantly reduces or even may substantially eliminate human errors, may improve the level of patient care, saves time and reduces stress and workload of the healthcare facility staff. According to some aspects, the disclosed systems and methods provide two levels of data validation, the first may be based on the patient’s clinical condition, e.g., a clinical profile, administrated medications and treatment plan, and the second may be performed by a medical professional, such as a nurse, followed by an automatic EMR reporting of the validated data.

According to some aspects, abnormal clinical trends may be identified based on data collected by the disclosed systems and methods from multiple resources, such as medical devices, sensors, and EMRs, to enable treatment plan adjustments and avoid hospital admissions. The abnormal clinical trends may be identified, for example, by utilizing artificial intelligence, or more specifically, deep learning techniques as known to a person skilled in the art.

In the following detailed description, specific details are set forth in order to provide a thorough understanding of the disclosure. However, it will be understood by those skilled in the art that the disclosure may be practiced without these specific details. In other instances, well-known methods, procedures, and components have not been described in detail so as not to obscure the present disclosure. Some features or elements described with respect to one system may be combined with features or elements described with respect to other systems. For the sake of clarity, discussion of same or similar features or elements may not be repeated.

Although the disclosure is not limited in this regard, discussions utilizing terms such as, for example, “processing,” “computing,” “calculating,” “determining,” “establishing,” “analyzing,” “checking,” or the like, may refer to operation(s) and/or process(es) of a computer, a computing platform, a computing system, or other electronic computing device, that manipulates and/or transforms data represented as physical (e.g., electronic) quantities within the computer’s registers and/or memories into other data similarly represented as physical quantities within the computer’s registers and/or memories or other information non-transitory storage medium that may store instructions to perform operations and/or processes.

Although the disclosure is not limited in this regard, the terms “plurality” and “a plurality” as used herein may include, for example, “multiple” or “two or more.” The terms “plurality” or “a plurality” may be used throughout the specification to describe two or more components, devices, elements, units, parameters, or the like. Unless explicitly stated, the methods described herein are not constrained to a particular order or sequence. Additionally, some of the described methods or elements thereof can occur or be performed simultaneously, at the same point in time, or concurrently.

The terms “measurement” as used herein may include or may be associated with a single value only for evaluation of a single vital sign or a single health indicator, while a single medical measurement device (will be also referred to herein as “medical device” or “measurement device”) may provide a plurality of measurements providing a plurality of corresponding values.

Although the disclosure is not limited in this regard, the terms “patient” and “patients” as used herein with respect to a healthcare facility may also refer to a resident or residents, respectively, of a healthcare facility.

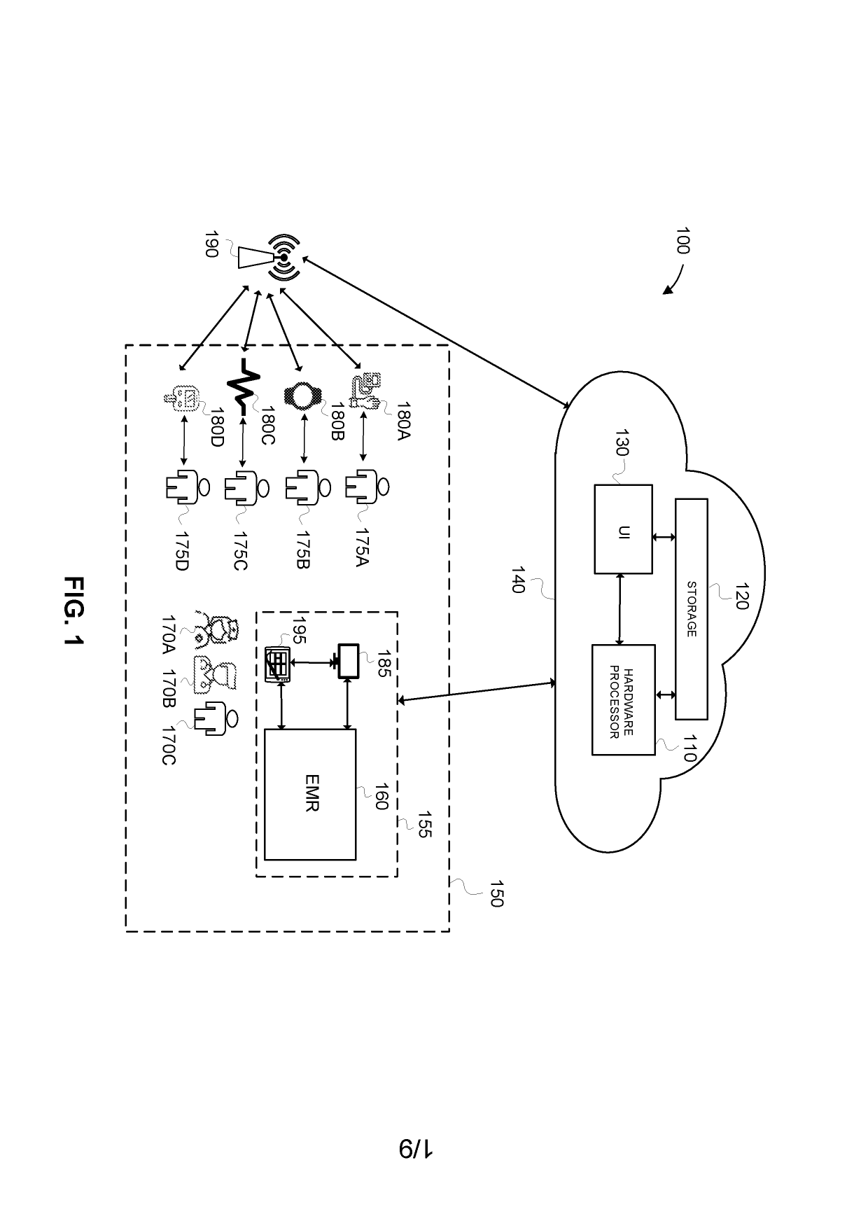

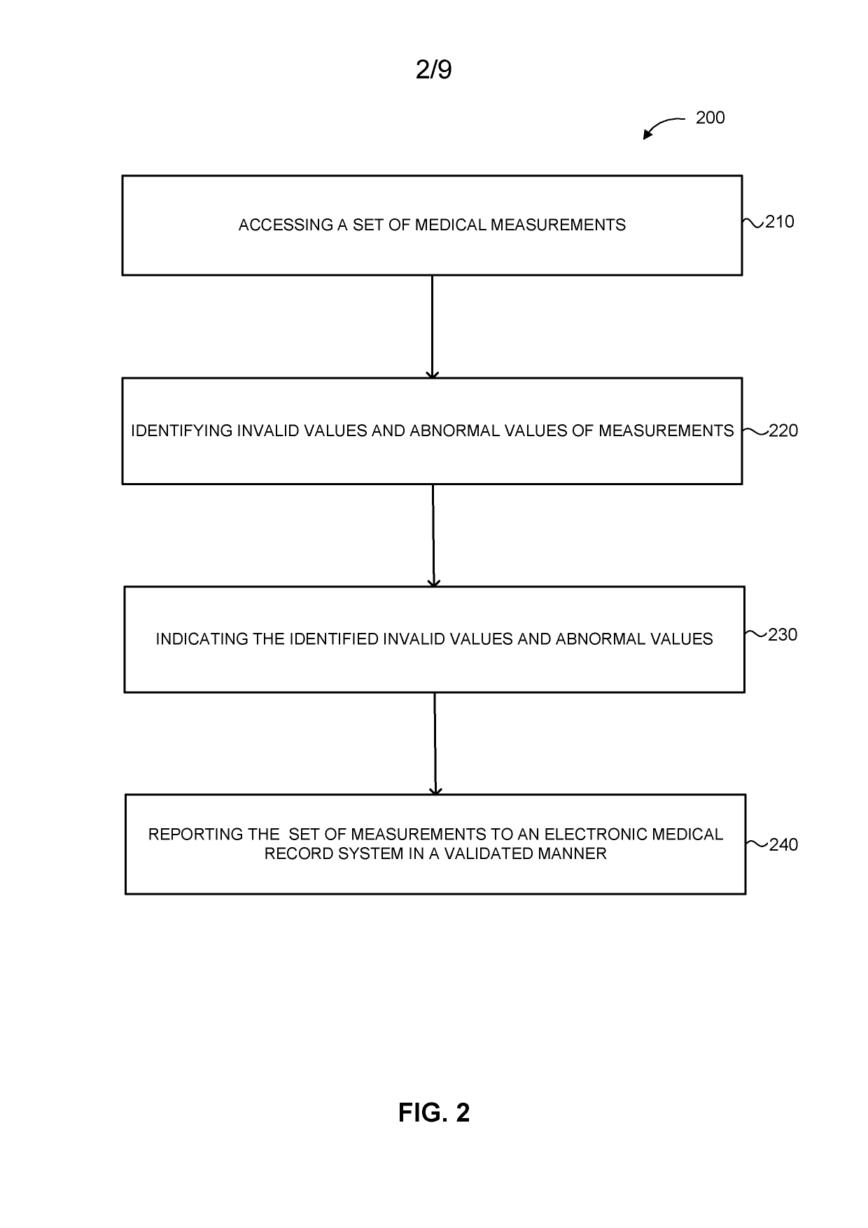

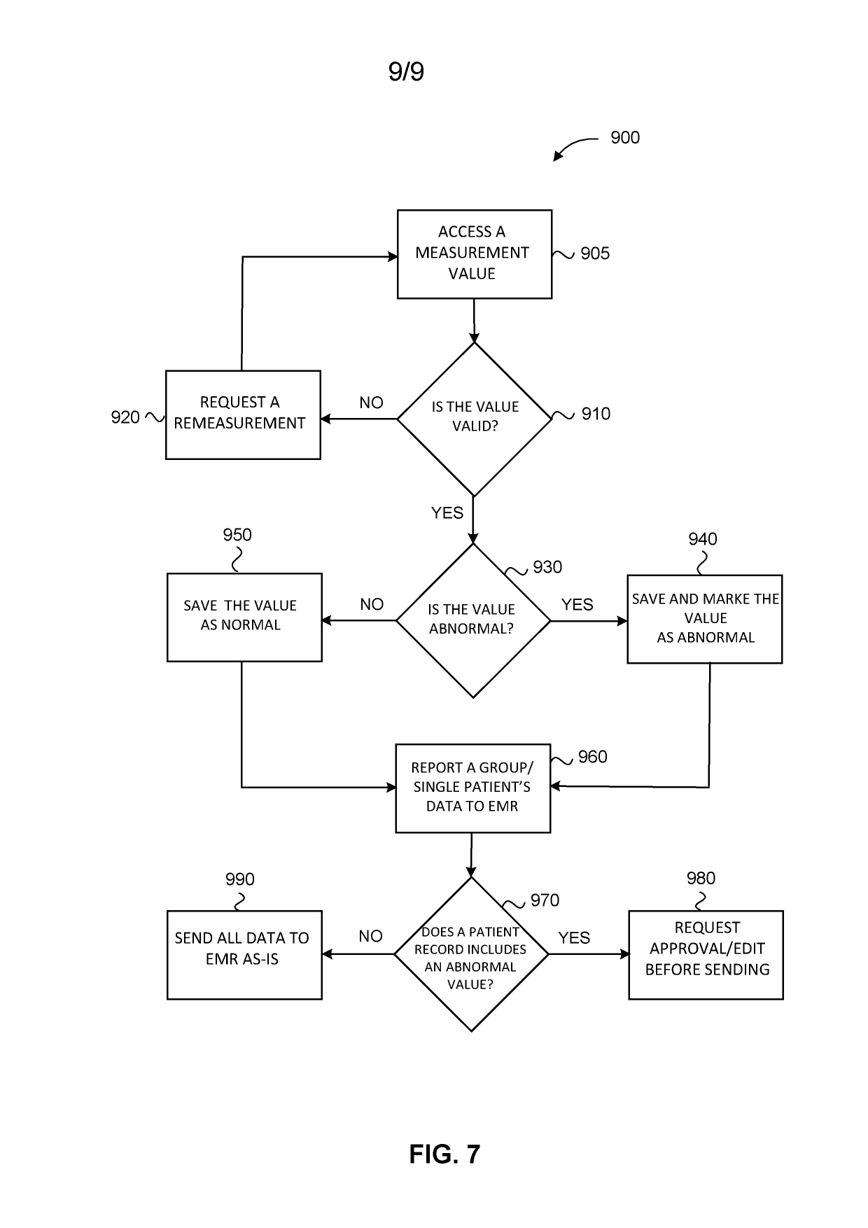

Referring to FIG. 1, there is shown a diagram of a system 100 for patient monitoring and validated EMR reporting for a healthcare facility 150. System 100 may include a hardware processor 110, a computer readable storage device 120 and a User Interface 130. Storage device 120 may store instructions for execution by hardware processor 110, e.g., to generate or operate UI 130. The instructions may further include instructions for executing method 200 of FIG. 2 or method 900 of FIG. 7. According to some aspects, system 100 may further include a communication device 190. System 100 may be in communication with healthcare facility 150, or with components thereof. System 100 may reside on one or more cloud infrastructures 140.

Healthcare facility 150 (will be also referred herein as “facility 150”) may include a computing system 155 which will be referred herein as Healthcare Facility computing system 155 or HF computing system 155. HF computing system 155 may be a local system or may be at least partially a remote system, e.g., by including cloud-based computing resources or cloud-based components. HF computing system 155 may include an EMR system 160, one or more display devices such as display 185 and one or more mobile computing devices such as a tablet 195. EMR system 160 may be a cloud-based system. The one or more mobile computing devices may include for example, one or more tablets such as tablet 195, or one or more iPads, smartphones etc. or a combination thereof. EMR system 160, display 185 tablet 195, and optionally measurement devices such as devices 180A-180D or a portion thereof, may communicate, for example, via a Local Area Network (LAN) or via the internet. Display 185 (e.g., of a terminal or of a desktop computing system) and tablet 195 may be used by the staff of facility 150, such as caregivers 170A-170C. Mobile computing devices such as tablet 195 may be used by the staff of the facility, e.g., for one or more of entering, reviewing, editing, approving, saving or reporting of measured values of health indicators, by that allowing on-spot measurement and EMR reporting. HF computing system 155 may be in communication with system 100, e.g., via UI 130.

EMR system 160 may be configured for recording or storing information relating to patients of facility 150, such as patients 175A-175D. System 100 may be in communication with EMR system 160 and may extract data referring to the patients of facility 150, such as patients 175A-175D. The extracted data or a portion of it may be stored in storage device 120. System 100 may additionally provide data, such as results of medical measurements (will be also referred to herein as “measurements”), e.g., provided by medical measurement devices such as devices 180A-180D, to EMR system 160 to be stored in EMR system 160. Devices 180A-180D may include or may provide dedicated software applications. Measurements data may be provided to system 100, e.g., via communication device 190, via staff of facility 150 such as caregivers 170A-170C, via HF computing system 155 or via a combination thereof.

Patients 175A-175D are residents of or at least temporarily staying at facility 150 and treated by or receive care or assistance from facility 150 staff, such as caregivers 170A-170C. Care givers 170A and 170B are Health Care Professionals (HCPs). An HCP provides healthcare treatment and advice based on formal training and experience and may be, for example, a nurse, a therapist or a physician. For example, caregiver 170A is a nurse (e.g., a certified nurse or a registered nurse) and caregiver 170B is a physician. Caregiver 170C is Unlicensed Assistive Personnel (UAP). A UAP assists patients such as patients175A-175D under the supervision of an HCP and does not hold a license or other mandatory professional requirements for practice. A UAP may be, for example, a patient care assistant, a Certified Nursing Assistant (CNA) or an orderly.

Caregivers 170A-170C may interact with, receive information from or provide information to system 100, e.g., with respect to patients 175A-175D, devices 180A-180D or with respect to their daily tasks, via UI 130. UI 130 may be a web-based UI. UI 130 may include a Graphical User Interface which may be displayed on a display device of facility 150 such as display 185. Display 185 may be a part of a computing system of facility 150. According to some aspects, at least a portion of Care givers 170A-170C may interact with EMR system 160, e.g., via display 185.

Medical measurement devices 180A-180D (will be also referred herein as “measurement devices”) may include monitors, sensors etc. employing various technologies for measuring or evaluating one or more health indicators such as vital signs, physiological indicators, body posture, position or movement or a combination thereof. Measurement or detection of body posture, position or movement may be used, for example, for fall prevention, pressure ulcer prevention, making sure that a patient is resting or not wondering or walking while using a walking assisting device. Vital signs may include blood pressure, heart rate, blood oxygen saturation level, glucose level and the like. According to some aspects, a measurement device may perform a continuous measurement, e.g., monitoring per a predefined time interval, or a non-continuous measurement, such as disperse or on-demand measurements. According to some aspects, a measurement device may be stationary, e.g., placed in a specific room or location of the healthcare facility, or portable, e.g., carried on a cart or wearable.

According to some aspects, the measurement (e.g., the result of the measurement and optionally additional data relating to the measurement, will be also referred herein as “measurement data”) may be provided automatically, semi-automatically, or manually. An automatic provision of measurements may include provision of the measurement to system 100, HC computing system 155 or both without requiring human intervention. A semi-automatic provision of measurements may include providing a preview of the measurement to a staff member of facility 150, such as caregivers 170A-170C, while the measurement data is sent to system 100 or to HC computing system 155 upon approval of the staff member. A manual provision of measurements may include the manual entering of the measurements data to an interface of system 100 (e.g., via tablet 195 or an I/O device coupled with display 185 and UI 130) or to an interface provided by HC computing system 155. Measurement device 180A measures blood pressure. Measurement device 180B is a smartwatch configured to measure a plurality of vital signs such as heart rate, respiratory rate and blood oxygen saturation. Measurement device 180C measures heart rate. Measurement device 180D measures blood glucose level. According to some aspects, measurement devices 180A-180D or a portion of them may be coupled with communication device 190 and may provide measurement values to system 100 via communication device 190. According to some aspects, measurement devices 180A-180D or a portion of them may be coupled with HF computing system 155. According to some aspects, the measurements data provided by measurement devices 180A-180D or a portion of them is provided to HF computing system 155 or to system 100 or both via one or more of caregivers 170A-170C, e.g., in case measurement devices 180A-180D or a portion of them are manual measurement devices. According to some aspects, system 100 may be provided with the measurements performed by measurement devices 180A-180D or a portion of them via HF computing system 155.

Reference is now made to FIG. 2, which is a flow diagram of a method for validated reporting to a health record system such as an EMR system. Method 200 may be applied by the disclosed systems, such as system 100 of FIG. 1. In a step 210, a set of measurements of one or more health indicators, e.g., vital signs, for one or more patients is accessed, where the set of measurements includes values of measurements of the set of measurements.

According to some aspects, the set of measurements is stored in a storage device of the disclosed systems, such as storage device 120 of system 100. According to some aspects, the values of the measurements may be stored in the storage device except for values which are invalid. In such case, invalid values are not saved by system 100. The invalid values may be kept in a temporary memory for review by a user or for the purpose of performing validation as disclosed herein (e.g., by replacing the invalid value by a valid value).

In a step 220, invalid values of measurements and abnormal values of measurements may be identified in the set of measurements. According to some aspects, identifying invalid values of measurements of the set of measurements includes determining a value of a measurement as invalid if the value is not within a predefined range. According to some aspects, the range may be defined by setting a minimum threshold and a maximum threshold. The range may be defined by the healthcare facility (e.g., facility 150) or based on the specific healthcare facility requirements or needs. The extent of the range may determine the form of the invalid value filtering, For example, a relatively narrow range may require a more subtle or more minded filtering. According to some aspects, the identifying of the abnormal values of the measurements may include evaluating each value of the values of the measurements based on available information about the patient from whom the respective measurement was taken. According to some aspects, the evaluation may be based on one or more of or a combination of: the medical condition of the patient (e.g., chronic diseases) or other attributes such as age, gender, origin etc. of the patient. Such information may be provided, for example, by an EMR system such as EMR system 160.

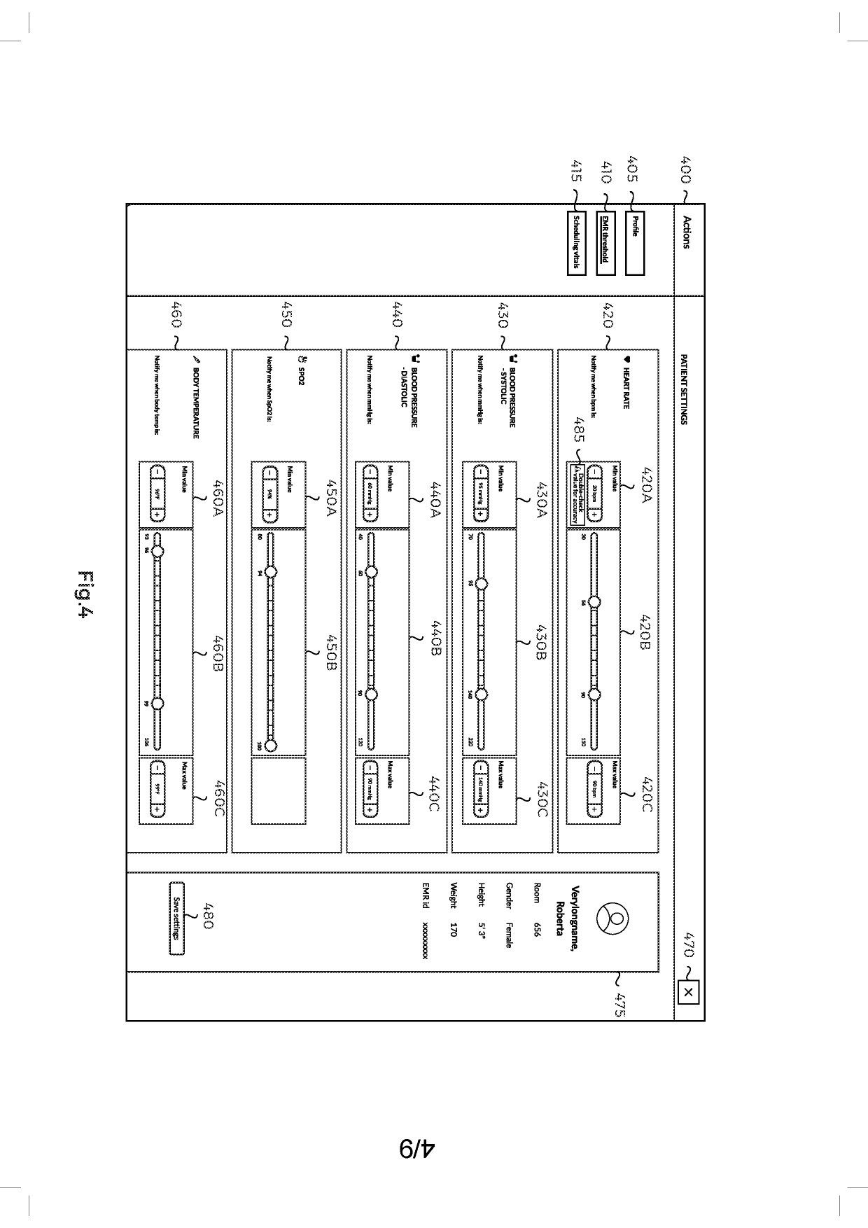

According to some aspects, each patient is associated with a predefined profile of a set of predefined profiles. Each profile of the set of predefined profiles may include one or more ranges of values of one or more measurements, e.g., of vital signs. Each range of values of the one or more ranges of values is defined for a different type of measurement (e.g., blood pressure, glucose level etc.). The profiles may be provided by the disclosed systems and methods and/or may be defined by facility 150. According to some aspects, a range may be determined by setting a minimum threshold and a maximum threshold, as shown, for example, in FIG. 4 and as will be elaborated hereinbelow.

According to some aspects, the evaluating of each value of the values of the measurements of the set of measurements may include determining a value of a measurement of the set of measurements as abnormal if the value of the respective measurement is not within the range of values defined for the respective measurement in the predefined profile associated with the patient from whom the respective measurement was taken. According to some aspects, editing of the predefined set of profiles is allowed. For example, professional staff of the facility such as physician 170B of facility 150 may be provided with permissions to edit a predefined profile to be more suitable to a specific patient or to generate new such predefined profiles.

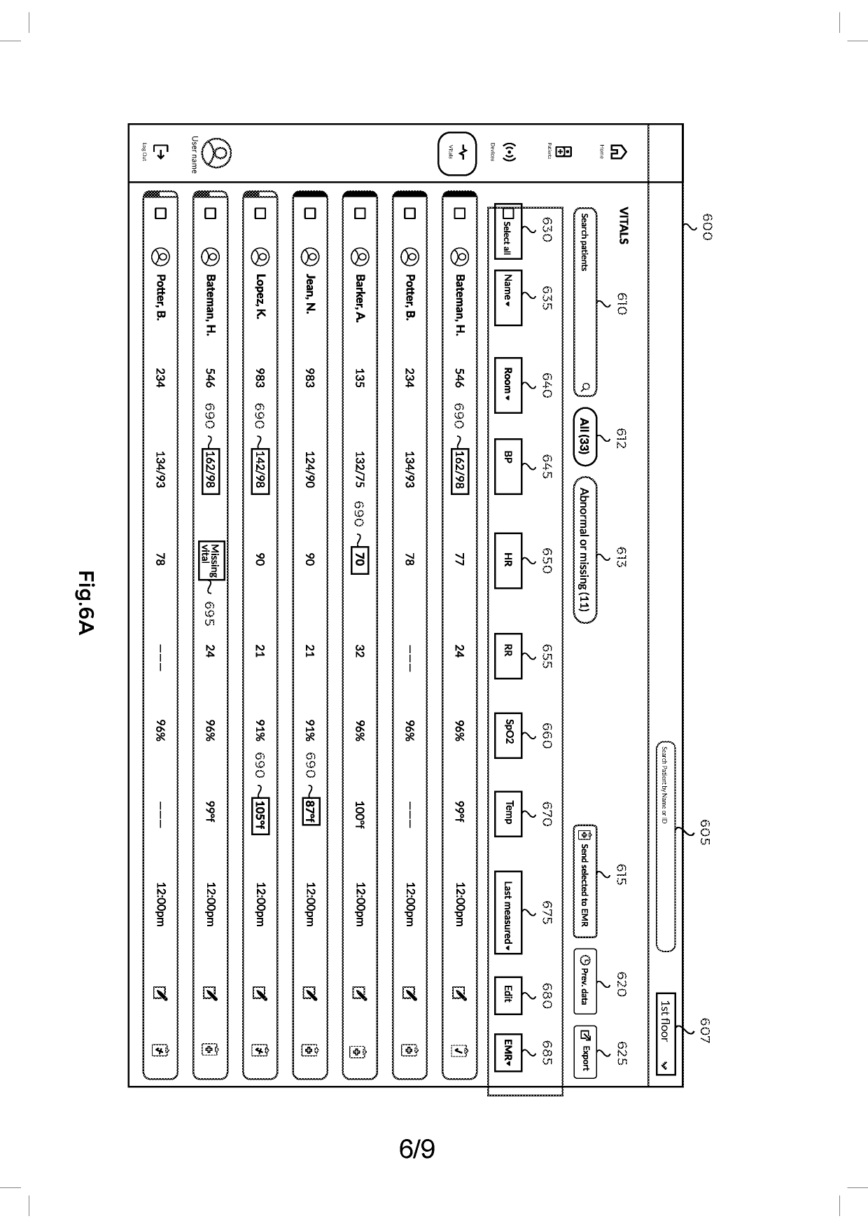

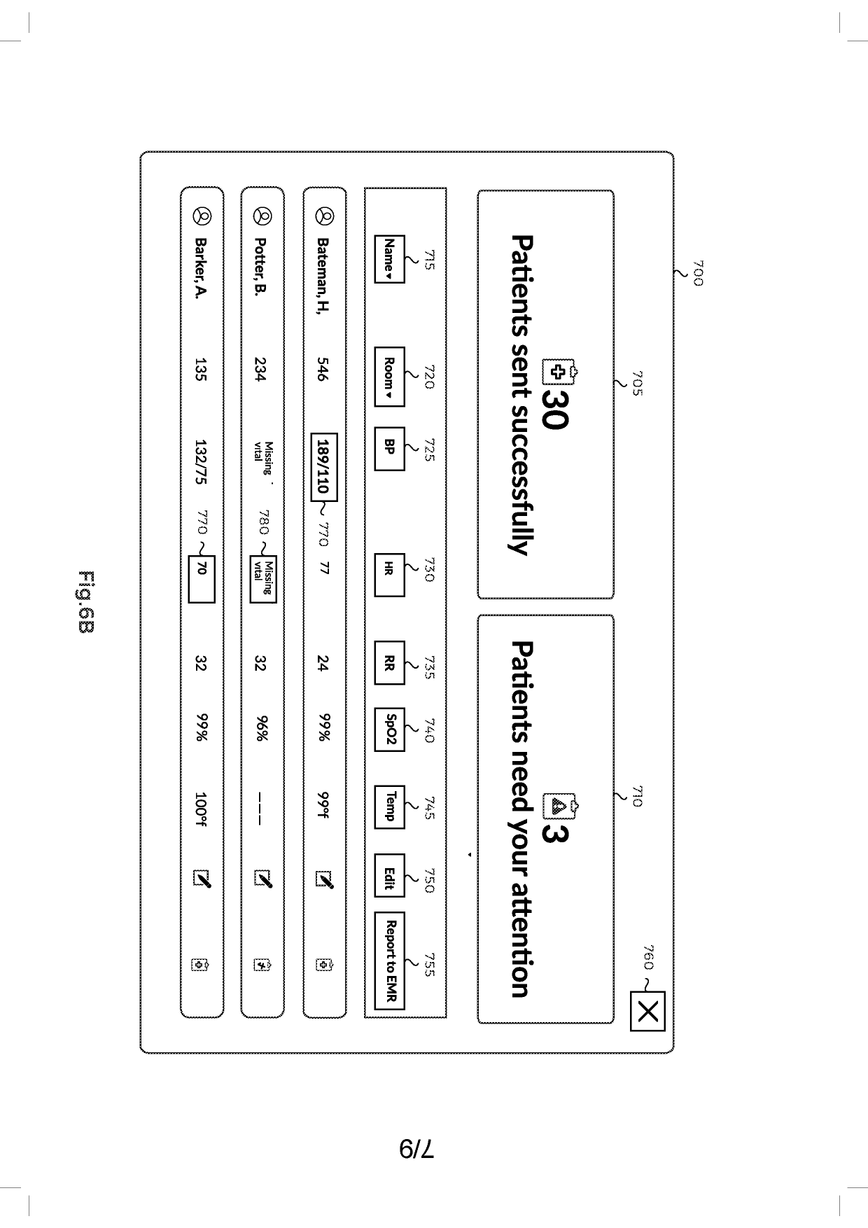

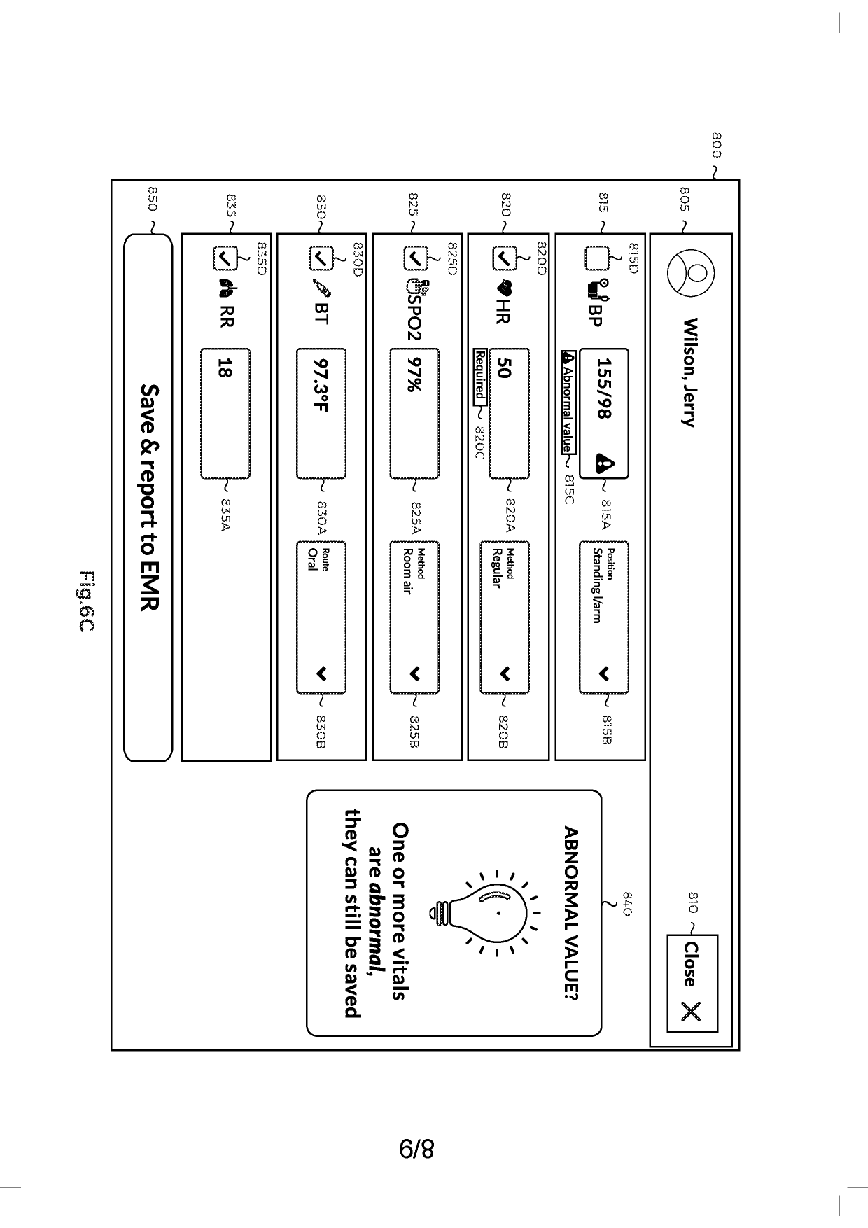

In a step 230, the identified invalid values of measurements and the identified abnormal values of measurements may be indicated, e.g., to a user. According to some aspects, indicating the identified invalid values of measurements and the identified abnormal values of measurements includes displaying, on a display device, the set of measurements where the identified invalid values and the identified abnormal values are visually indicated. The visual indication may include for example, a mark, an icon or color coding. With reference to FIG. 1, staff members of facility 150, such caregivers 170A-170C, may review such visual indications on display 185 or tablet 195. A page of a GUI including such an indication is shown, for example, in FIG. 6A, as will be elaborated hereinbelow. According to some aspects, invalid values are not indicated, for example, when invalid values are automatically filtered.

In a step 240, the set of measurements may be reported to an EMR system in a validated manner. Reporting the set of measurements to the EMR system in a validated manner may include reporting a portion of the set of measurements or the entire set of measurements. A portion of the set of measurements may be reported if only a portion of the set of measurements is validated. The validation may include editing an invalid value and editing or approving an abnormal value. According to some aspects, the validation may include editing or filtering an invalid value and editing or approving an abnormal value.

For each measurement having an invalid value, the measurement may be reported to the EMR system (e.g., EMR system 160) only after a valid value is received for the measurement and the invalid value is replaced by the valid value. According to some aspects, invalid values may be automatically filtered. For each measurement having an abnormal value, the measurement may be reported to the EMR system only after the abnormal value is approved or a normal value is received for the measurement and the abnormal value is replaced by the normal value.

According to some aspects, users authorized by the healthcare facility, e.g., staff members of facility 150, like caregivers 170A-170C, may have one or more permissions assigned to them for using or utilizing the disclosed systems and methods. The permissions may allow different levels of use or utilization of the disclosed systems and methods, e.g., according to the user’s qualification or role within the healthcare facility. The permissions may refer, for example, to the ability to edit invalid and/or abnormal and/or confirm abnormal measurement values.

According to some aspects, if an invalid value is included in the accessed set of measurements, a user may be allowed to edit the invalid value, if he has an adequate permission. If the user does not have an adequate permission, the user may be requested to repeat the respective measurement. For example, a user who is a UAP, like user 170C, may be provided with a lower level of permissions which do not allow editing invalid measurement values or editing measurement values in general. As opposed to that, a user who is an HCP, like users 170A and 170B, may be provided with a higher level of permissions which allow the user to edit an invalid value or edit values in general.

According to some aspects, approval of an abnormal value of a measurment of the set of measurments is allowed only by a user who has an adequate permission, e.g., an HCP such as caregivers 170A and 170B. According to some aspects, editing an abnormal value of a measurement is allowed only by a user who has an adequate permission, e.g., an HCP such as caregivers 170A and 170B.

According to some aspects, the reporting of the set of measurements to the EMR system may be performed in a batch. According to some aspects, the reporting of the set of measurements to the EMR system may be performed individually, e.g., per patient or per measurement. According to some aspects, the set of measurements, e.g., of one or more vital signs, may be continuously updated. According to some aspects, the reporting to the EMR system may be performed according to one or more predefined rules. For example, a continuously updated measurement may be reported at predefined times. According to another example, one or more measurements may be defined as mandatory (e.g., per a patient or per an assigned profile) and a set of measurements may not be reported unless it includes the predefined mandatory measurements. According to some aspects, the reporting to the EMR system may be performed at predefined times.

At times, one or more measurements may be missing a value. This may happen, for example, when a measurement failed, a measurement was not performed or when a caregiver did not enter a value for the measurement. According to some aspects, measurements of one or more health indicators may be defined as an essential type of measurement. According to some aspects, definition of a measurement as an essential measurement may be for all patients of the healthcare facility, per specific type of patient (e.g., having a specific medical condition), per specific patients or a combination thereof. According to some aspects, for each measurement of the set of measurements missing a value and of a type defined as an essential type of measurement, the reporting of the respective measurement to the EMR system may be performed only after the lack of value is approved or a value is received for the respective measurement. According to some aspects, the approval of a lack of value or of a missing value may be performed only by a user who has an adequate permission, e.g., an HCP such as caregivers 170A and 170B. According to some aspects, a missing value may be also indicated to a user, e.g., visually.

According to some aspects, the value for the respective measurement may be received by allowing a user who has an adequate permission, e.g., an HCP like caregivers 170A and 170B, to enter the value. This may be achieved by allowing editing of the accessed or received measurements values. Accordingly, an invalid, abnormal or a missing value may be edited or entered, respectively.

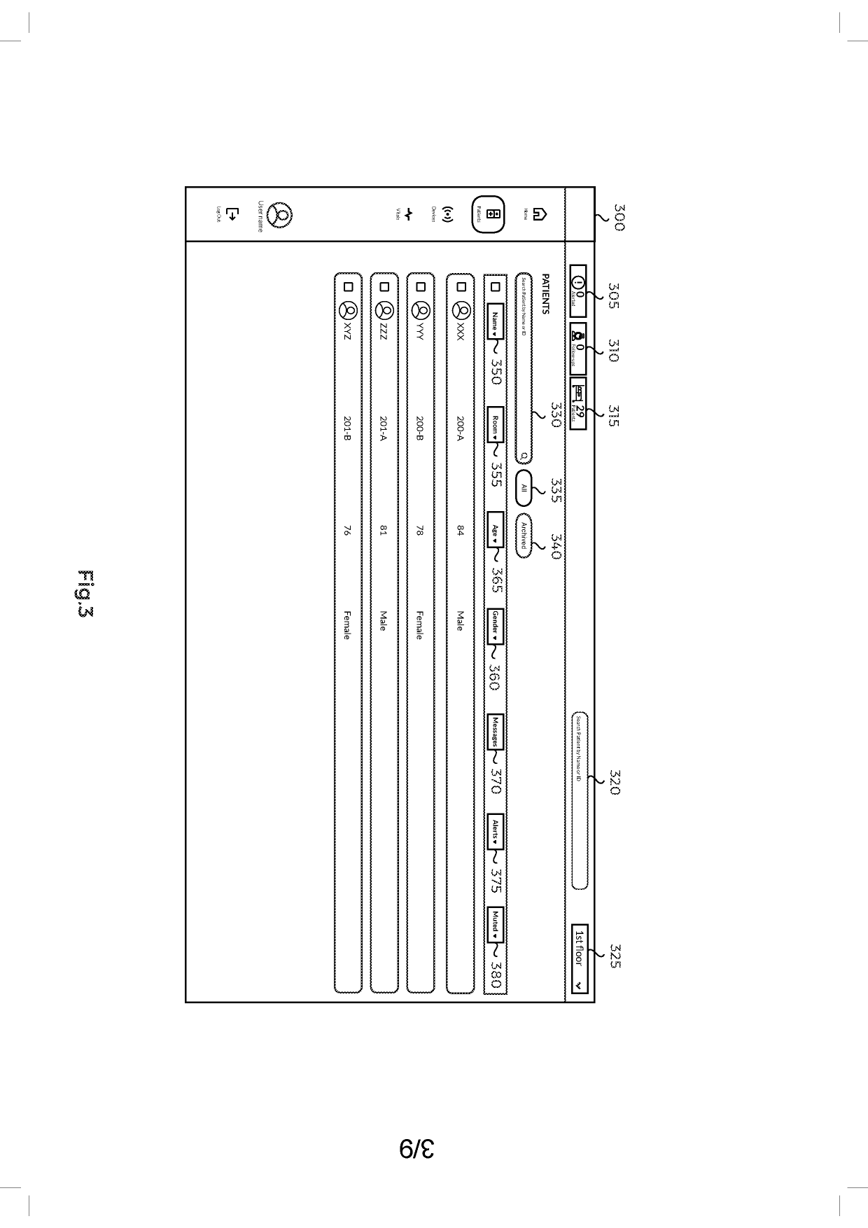

Reference is now made to FIG. 3, which is an illustration of a page 300 of a GUI presenting a list of patients in a healthcare facility. A UI of the disclosed system may include the GUI. Referring to FIG. 1, the GUI may be included in UI 130 of system 100. The GUI may be presented to caregivers such as caregivers 170A-170C on display 185 or tablet 195. Page or screen 300, which may be also referred to as the patients’ dashboard, may include one or more of the following indications: number of alerts 305, number of follow-up patients 310 or the total number of patients 315. Number of alerts 305 indicates the number of active alerts. Alerts may be defined for various scenarios, such as abnormal measurement values which were not edited or approved, missing values for essential measurements etc. Number of follow-up patients 310 indicates the number of patients which require follow-up. One or more criteria may be defined for determining a patient as a follow-up patient. Such criteria may be a patient having an acute condition, a patient having an abnormal measurement value of a vital sign, and the like. Page 300 may also include search boxes like search box 320 and 330. Search box 320 may allow indicating a searched patient in the dashboard while search box 330 may allow opening a pop-up window including the specific searched patient details or presenting his file or records. Search boxes 320 and 330 may allow searching for a patient by various attributes like name or identification number (ID). Page 300 may further include elements allowing filtering of the patients’ list like drop box 325, which allows presenting a list of patients which stay in a specific floor or click buttons 335 and 340, while click button 335 allows presenting all the patients (e.g., which stay at the specific selected floor) and click button 340 allows presenting all the archived patients. The disclosed systems (such as system 100) may synchronize with an EMR system of the healthcare facility (e.g., once in a predefined time interval) to keep an updated list of admitted and discharged patients (e.g., archived patients). The list of patients included in page 300 is presented in a table form. The table may include fields representing relevant information of the patient like a name field 350, a room field 355, a gender field 360 and an age field 365. The patients table may further include additional fields like a messages field 370, an alerts field 375 and a muted field 380. Messages field 370 may include messages added by the staff of the facility, typically professional staff like caregivers 170A and 170B of facility 150 for each patient in the list. Alerts field 375 may indicate the pending alerts for each patient in the list. Muted field 380 may include indication if the alerts are muted for the respective patient. Alerts may be muted, for example, when medication was administrated to a patient in response to a measured abnormal value of a vital sign. The abnormal value alert may be then muted for a predefined number of hours, allowing the treatment to take its course.

According to some aspects, the disclosed systems and methods may provide an automatic patient admission. With reference to FIG. 1, a patient record may be created in system 100 once the patient is admitted in EMR system 160 of facility 150. System 100 may automatically identify a new patient admitted in EMR system 160. The patient records may be stored in a database of system 100. The database may be stored in storage device 120. This may be performed by fetching the patient data from EMR system 160, creating the patient in the list of patients of system 100, including patient details such as name, gender, room, image, facility, unit etc., as shown, for example, in FIG. 3. According to some aspects, the patient's clinical data (e.g., medical conditions) may be scanned in EMR system 160 to allocate a profile and thresholds for health indicators such as clinical or physiological data (e.g., vital signs), as detailed herein.

According to some aspects, the disclosed systems and methods may provide an automatic patient discharge including suspension of the patient record in the disclosed system database when the patient is discharged in the EMR system of the healthcare facility. The patient may be then moved from an active patients’ list to archived patients’ list. Furthermore, the disclosed systems and methods may disassociate measurement devices that were allocated to the dismissed patient. According to some aspects, a patient may be deactivated. Deactivating a patient may stop the monitoring and task follow-up. A user (e.g., a caregiver such as professional caregivers 170A-170B) may deactivate a patient in case he/she is outside the facility, e.g., for treatment or with family, but still admitted.

The disclosed systems and methods may allocate or associate profiles and health indicators, such as vital signs, validation thresholds to a patient based on the patient’s medical conditions, e.g., as recorded in an EMR system. Several pre-defined profiles may be built or generated based on chronic characteristics common, e.g., in elders. When a patient is admitted to the disclosed systems (such as system 100 of FIG. 1), the system may scan his EMR record (e.g., in EMR system 160), identify his recorded conditions, and allocate a profile accordingly.

The patient’s profile may define the health indicators (e.g., clinical and physiological indicators) thresholds. These thresholds may determine if the measured health indicators (e.g., vital sign) are abnormal and output an indication or a notification (e.g., an alert) accordingly to the caregivers or the clinical team (e.g., caregivers 170A-170C of FIG. 1). According to some aspects, the thresholds and profile may be edited via the patient’s profile page.

Reference is now made to FIG. 4, which is an illustration of a page 400 of the GUI of FIG. 3 for editing thresholds of a profile assigned to a patient of the healthcare facility, such as patients 175A-175D of facility 150. Page 400 may be, for example, a pop-up window accessible from a patient page or dashboard. Page 400 may include a navigation menu including list of links to further pages such as a profile link 405, an EMR threshold link 410 (shown) and a scheduling vitals link 405. Profile link 405 may lead to a profile page including details on the patient’s assigned profile. EMR threshold link 410 is shown (leads to page 400 and indicated accordingly) which allows setting or editing the assigned profile. Scheduling vitals link 405 allows determining a schedule for measurement of a selected vital sign (e.g., continuous per a defined time interval or non-continuous at predefined times). As shown, the assigned profile includes thresholds for a number of vital signs: heart rate 420, systolic blood pressure 430, diastolic blood pressure 440, Saturation of Peripheral Oxygen (SpO2) 450 and body temperature 460. Each health indicator (in this example, vital signs) includes a text box for entering a value for a minimum threshold (if applicable), like text boxes 420A, 430A, 440A, 450A and 460A, respectively; a text box for entering a value for a maximum threshold (if applicable), like text boxes 420C, 430C, 440C and 460C, respectively; and a slide bar for entering the range of values like slide bars 420C, 430B, 440B, 450B and 460B, respectively. Page 400 may also include an alert UI element 485 which appears, for example, when an invalid value is entered in one of the text boxes. Page 400 may additionally include a patient details element 475 which may include a picture of the specific patient and relevant information like room, gender, height, weight and EMR ID; a save button 480 allowing to save the settings of the assigned profile and a close window element 470 for closing the pop-up window.

Connectivity with third party measurement devices such as measurement devices 180A-180D of FIG.1 may be performed via various technologies and may depend of the configuration of each device. According to some aspects, connectivity may be achieved by cloud-to-cloud integration. Cloud-to-cloud integration may be implemented, for example, by directly calling the measurement device associated cloud services to pull data and push configuration or by receiving the measurement device associated webhooks and querying the measurement device associated services.

According to some aspects, the disclosed system and methods may use a direct connection to the measurement device. The measurement device may send data to the disclosed systems over the internet. Other methods and technologies, such as utilizing Software Development Kits (SDKs), may be used as known to a person skilled in the art.

According to some aspects, existing devices or new measurement devices may be associated with patients to allow monitoring of the patient's data or health indicators (e.g., vital signs, location or posture). Once the measurement device is associated, the data collected from the measurement device may be saved in the database of the disclosed systems (e.g., in storage device 120 of system 100 of FIG. 1).

According to some aspects, the GUI (such as a GUI of UI 130 of system 100) may include a device management dashboard accessible to the users. The dashboard may present which measurement devices are associated with whom of the patients. According to some aspects, a patient page or dashboard may present the measurement devices that are associated with the respective patient.

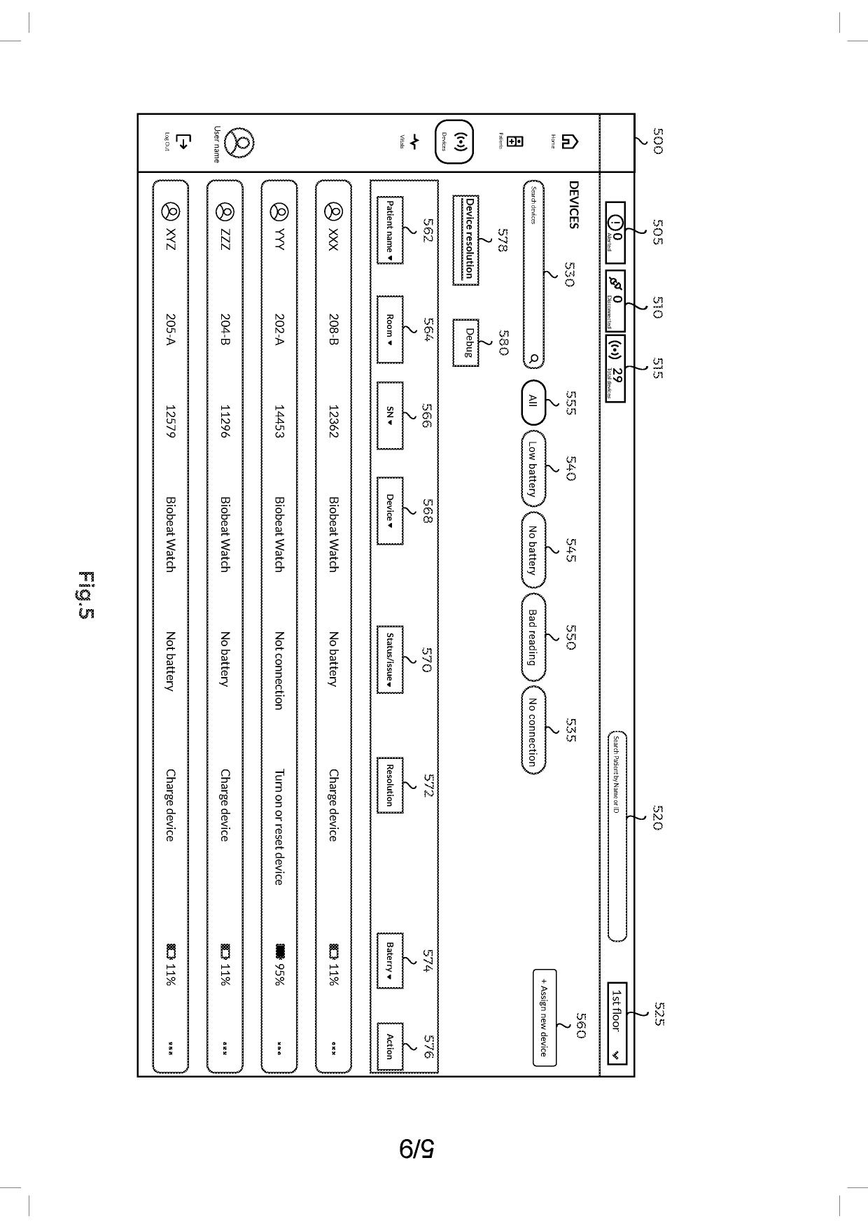

Reference is now made to FIG. 5, which is an illustration of a page 500 of the GUI of FIG. 3 presenting the status of medical devices assigned to patients of the healthcare facility and in communication with the system of FIG. 1. Page or screen 500, which may be also referred to as the measurement devices dashboard includes a list or a table showing measurement devices which require technical resolution or debugging according to the assigned patient. Page 500 may also include one or more of the following indications: number of alerted devices 505, number of disconnected devices 510 or the total number of devices 515. Number of alerted devices 505 indicates the number of devices having active alerts, e.g., due to technical issues. Number of disconnected devices 510 indicates the number of devices which are disconnected. Page 500 may also include search boxes like search box 520 and 530. Search box 520 may allow searching for a specific patient and presenting his dashboard. Search box 530 may allow searching for a specific device and indication of the search device in the list of devices. Search boxes 520 and 530 may allow searching for a patient or a device by various attributes. Page 500 may further include elements allowing filtering of the devices’ list like drop box 525, which allows presenting a list of devices which are located at a specific floor or carried by patients which stay at the specific floor, or click buttons 535, 540, 545, 550, and 555. Click button 555 allows presenting all the devices (e.g., which are located at the selected specific floor or carried by patients which stay at the selected specific floor). Click button 535 allows presenting all the devices which have no connection to, e.g., to system 100 of FIG. 1. Click button 540 allows presenting all the devices which have low battery. Click button 545 allows presenting all the devices which have no battery. Click button 550 allows presenting all the devices which have a bad reading.

Page 500 shows a table of devices which require resolution 578 according to their assigned patients. Page 500 includes a navigation menu which allows selecting between device resolution table 578 and debugging table 580. The table may include fields representing relevant information relating to the devices like a patient name field 562 (e.g., the name of the patient the device is assigned to), a room field 564 (e.g., the room the device in which is located or the room in which the patient carrying the device stays), a Serial Number (SN) field 566 (e.g., the SN of the device), a devices field 568 (e.g., the name of the device), a status/issue field 570 (e.g., the issue of the device which require attention or resolution), a resolution field 572 (e.g., detailing the type of resolution required like charge device or reset device), a battery field 574 (e.g., indicating battery power left) and an action field 576. The action field may include reference to a menu allowing performing various actions like de-assigning a device or calibrating a device.

Page 500 also includes a drop box 560 allowing the user to assign a new device to a patient. Once a user selects to assign a new measurement device, the user may choose a device from a list of supported devices associated with the healthcare facility, and optionally to a specific unit of the healthcare facility and assign it to a patient in this unit. According to some aspects, a user may view the list of measurement devices associated with a specific patient and their status in the patient dashboard.

According to some aspects, a user may disassociate existing devices to stop monitoring a patient. This may be performed from the specific device page or from the specific patient page or dashboard.

Some measurement devices may require calibration to provide accurate measurements. Calibration is usually done after a device is associated with a patient. The disclosed systems and methods may include a wizard which guides the staff of the facility in the process of calibration to ensure it is done properly. According to some aspects, and to avoid providing wrong data, the disclosed systems and methods may not allow monitoring of a patient, even if the measurement device is already associated with the patient, as long as the measurement device is not calibrated. According to some aspects, an indication that the patient's measurement device is not calibrated may be provided to the user. Once the calibration is complete, and the measurement device enters the monitoring mode, it may be indicated to the user (e.g., by color coding). The indication may be presented in the devices’ dashboard, the patients’ dashboard or in a health indicators report.

According to some aspects, and as shown in field 570, the device status may be presented, such as: “Not calibrated”, indicating the device needs to be calibrated to start monitoring the patient; “Low battery”, indicating the device needs to be placed in the charger; “No battery”, indicating the device has no battery left and stopped reporting; and “Bad Reading”, indicating the device has not managed to collect accurate data. According to some aspects, the disclosed systems and methods may offer a resolution for each status, to assist the user in operating the devices.

FIG. 6A is an illustration of a screenshot of a page 600 of the GUI of FIG. 3 presenting a table of values of measurements of vital signs to be reported to an EMR system. Page 300 includes a table of a set of measurements, or the set of the current measurements, of vital signs to be reported to an EMR system, such as EMR system 160 of facility 150 of FIG. 1. Page 600 may further include elements allowing filtering of the measurements’ list like drop box 607, which allows presenting a list of measurements of patients which stay in a specific floor or click buttons 612 and 613, while click button 612 allows presenting measurements of all the patients (e.g., which stay at the specific selected floor) and click button 613 allows presenting all the measurements having abnormal or missing values. Page 300 further includes search boxes like search box 605 and 610 which are similar to search boxes of page 300 of FIG. 3. Page 300 may additionally include elements allowing command buttons 615, 620 and 625. Command button 615 sends the selected records of the table of measurement values, which are validated according to the disclosed systems and methods, to the EMR system, upon activation. Command button 620 may present previous measurement values data (also known as “history”) upon activation. Command button 625 may allow export of the presented or current set of measurements, e.g., by converting it to PDF format.

The table of the set of measurements may include fields representing relevant information like a name field 635, for the patient whom the measurement was taken from, a room field 640 indicating the room which the patient is staying at and measurement value fields 645-670, for HR, RR, SpO2 and Temp for body temperature, respectively. The table further includes a “Last measured” field 675 for indicating the time of the last measurement, an “Edit” field 680 for editing the measurements values and an “EMR” field 685 indicating the status of EMR reporting. The optional statuses may be reported, not reported or partially reported, e.g., when not all the required health indicators or specifically as exemplified here, vital signs, were reported. The table may further include a field 630 including check boxes for selecting all the records or specific records. For example, only the selected records may be sent to the EMR system upon activation of command button 615. Solid line rectangles 690 indicate abnormal values such as BP 162/98, HR 70 or Temp 87℉. Other visual indications may be used such as color coding (e.g., red for abnormal values). Missing values for essential measurements are indicated by adding missing value text box 695 “Missing vital” (will be also referred as missing value 695) instead of the missing value. A user having an adequate permission may use the edit option 680 to enter a normal value instead of the abnormal values (following a remeasurement or by correcting a typo, for example) and may complete the missing value 695 (e.g., by performing the measurement, remeasuring or entering the missed value). The set of measurements shown in page 600 does not include invalid values which may be validated by a user (e.g., by remeasuring or by editing) in a prior stage or may be automatically filtered.

FIG. 6B is an illustration of a page 700 of the GUI of FIG. 3 presenting a status report following an EMR reporting action. Page 700 may be a pop-up window received after a set of measurements is reported to the EMR system (e.g., by activating command button 615 of page 600). Page 600 includes a table of a subset of measurements of a reported set of measurements which include abnormal values that were not attended and therefore were not reported. Page 300 further includes indications 705 and 710. Indication 705 indicates the number of patients (which may be also referred to as the number of measurements or records) that were reported successfully to the EMR system. Indication 710 indicated the number of patients which were not reported to the EMR system successfully and require the user’s attention. In the specific example of FIG. 6B, 30 patients were successfully reported while three patients were not. These patients are listed in the shown table. The table includes fields representing relevant information, corresponding to fields of the table of FIG. 6A, like a name field 715, a room field 720, measurement value fields 725-745, for HR, RR, SpO2 and Temp, respectively, an “Edit” field 750 and an “EMR” field 755. Rectangles 770 indicate abnormal values BP 189/110 and HR. Missing value for HR, which is defined as an essential measurement for the respective patient (Potter, B.) is indicated by adding missing value text box 780 “Missing vital” instead of the missing value. A user having an adequate permission may use the edit option to enter a normal value instead of the abnormal values (following a remeasurement or by correcting a typo, for example) and may complete the missing value (e.g., by performing the measurement, remeasuring or entering the missed value). Page 700 further includes an element 760 for closing pop-up window 700.

FIG. 6C is an illustration of a page 800 of a GUI designed for a mobile computing device, such as tablet 195 of FIG. 1, presenting values of measurements of vital signs for on-¬spot handling. Page 800 shows a dashboard for a set of measurements 815-835 performed on-spot for a patient 805. Page 800 may be a pop-up window which may be closed by activating element 810. Measurement 815 is a BP measurement. Measurement 815 includes a text box 815A for automatically (e.g., by receiving the value automatically from a connected measurement device) or manually entering the measurement value, a dropdown box 815B for selecting the measurement position, an alert or notification 815C since the value entered in text box 815A is abnormal and a checkbox 815D. Measurement 820 is an HR measurement. Measurement 820 includes a text box 820A for automatically or manually entering the measurement value, a dropdown box 820B for selecting the method of measurement, an alert or notification 820C indicating that the measurement is an essential measurement for this patient and thus required and a checkbox 820D. Measurement 825 is an SpO2 measurement. Measurement 825 includes a text box 825A for automatically or manually entering the measurement value, a dropdown box 825B for selecting the method of measurement, and a checkbox 825D. Measurement 830 is a Body Temperature (BT) measurement. Measurement 830 includes a text box 830A for automatically or manually entering the measurement value, a dropdown box 830B for selecting the route of the measurement (e.g., oral or otic), and a checkbox 830D. Measurement 835 is an RR measurement. Measurement 835 includes a text box 835D for automatically or manually entering the measurement value and a checkbox 830D. A user having an adequate permission may edit the automatically entered values and specifically edit the invalid value entered in text box 815A. According to some aspects, a user may have permission to edit only a specific one or more health indicators. For example, a non-professional staff member of the healthcare facility may be permitted to edit a body temperature measurement. Page 800 additionally includes a notification 840 and a command button 850 allowing saving the values to the disclosed systems, such as system 100 of FIG. 1, and also reporting the measured values to the EMR for patient 805 if the entered values are validated, or the BP value in text box 815A in the specific example presented in FIG. 6C.

According to some aspects, the disclosed systems and methods may adhere to treatment protocols and regulations. A vitals collection protocol may be built or generated for every patient based on a physician’s orders, administrated medications, clinical conditions, time of admission, regulatory requirements, payer requirements and the like. It may be validated or confirmed that all vitals defined as to be mandatory reported to the EMR system, e.g., during each shift, are being delivered and a user notification may be generated in case of incompliance. The user that may be notified, accordingly, that vitals or specific vitals must be collected, and execution may be validated. For each patient, an indication may be provided to the user that vitals collection duties were fulfilled fully and accurately.

The disclosed systems and methods allow supervising an auto-sending by screening only vitals that are not within the predefined ranges or thresholds (e.g., invalid or abnormal vitals). As opposed to manual screening performed by an HCP, such as a nurse, the disclosed auto-screening or validation mechanism saves time and ensures the quality and accuracy of vitals. According to some aspects, enforced EMR login may be performed, before reporting to the EMR system, via the disclosed systems and methods without the need to access the EMR system, to ensure only qualified and authorized personnel report to the EMR system.

The disclosed systems and methods may allow a bi-directional communication with EMR systems. Patient’s vitals flow from the disclosed systems to the EMR system, and information such as demographics, personal details, vitals, conditions, medications, physicians' orders, and other information may be transferred to the disclosed systems, e.g., to storage device 120 of system 100 of FIG. 1.

According to some aspects, the disclosed systems and methods may allow a single-click reporting of vitals to an EMR system. The patient's vitals may be sorted, and with a single click, all the vitals within the defined thresholds (e.g., valis and normal) may be sent. For those that exceed the thresholds, an authorized user may be notified accordingly and be able to edit these vitals before sending or approving them as-is.

According to some aspects, the disclosed systems allow a user to screen the vitals before sending them to an EMR system. A fast and convenient screening may be provided by allowing sorting patients by various parameters such as room, vitals, measurement time or name and by providing a simple and clear UI to screen, edit and report the vitals.

According to some aspects, the disclosed systems and methods provide integration of devices which allow the collection of patient data manually (such as heart rate, temperature etc.) while acquiring it automatically to after screening and validating of the data is performed.

Reference is now made to FIG. 7, which is a flow diagram showing an exemplary procedure 900 for validated reporting to an EMR system. Procedure 900 is exercised according to method 200 of FIG. 2, unless indicated differently. Procedure 900 may be applied by the disclosed systems, such as system 100 of FIG. 1. Procedure 900 may be applied to each received measurement, such as measurements provided via devices 180A-180D. At a step 905, a measurement value is accessed. At a step 910, the value is determined to be valid or invalid. If the value is determined invalid, the user (e.g., caregiver 170A, 170B or 170c) is requested to remeasure the value at a step 920. If the value is determined valid, the value is checked for abnormality at a step 930. If the value is determined abnormal, the value is saved, e.g., to storage 120 of system 100 and marked as abnormal according to step 940. If the value is determined normal (e.g., is not abnormal), then the value is saved, e.g., to storage 120 of system 100 as a normal value (e.g., not marked, indicated or flagged as abnormal). At a step 960, a record data of a single patient including one or more measurement values is reported to an EMR system such as EMR system 160. Alternatively, measurement data of a group of patients is reported. Prior to saving the measurement data in the EMR, each patient record is checked for abnormal values at a step 970. If an abnormal value is identified in a patient’s record, a user (e.g., caregiver 170A, 170B or 170C), e.g., depending on his/her permissions, is requested to approve the abnormal value or to edit the abnormal value, e.g., by replacing it with a normal value at a step 980. A patient record having an abnormal value will not be sent to the EMT until it is validated as disclosed herein. If the patient record does not have an abnormal value (e.g., all values are normal), the one or more patients’ records are sent to the EMR as a step 990.

The disclosed systems may include a processor or controller, such as hardware processor 110 of system 100 of FIG. 1, that may be or include, for example, one or more central processing unit processor(s) (CPU), one or more Graphics Processing Unit(s) (GPU or GPGPU), and/or other types of processors, such as a microprocessor, digital signal processor, microcontroller, programmable logic device (PLD), field programmable gate array (FPGA), or any suitable computing or computational device. The disclosed systems may also include an operating system, a memory, a storage, input devices, output devices, and one or more communication devices. A communication device may include one or more transceivers which allow communications with remote or external devices and may implement communications standards and protocols, such as cellular communications (e.g. 3G, 4G, 5G, CDMA, GSM), Ethernet, Wi-Fi, Bluetooth, low energy Bluetooth, Zigbee, Internet-of-Things protocols (such as mosquitto MQTT), and/or USB, among others.

The operating system may be or may include any code designed and/or configured to perform tasks involving coordination, scheduling, arbitration, supervising, controlling or otherwise managing operation of the system, such as scheduling execution of programs. The memory may be or may include, for example, one or more Random Access Memory (RAM), read-only memory (ROM), flash memory, volatile memory, non-volatile memory, cache memory, and/or other memory devices. The memory may store, for example, executable instructions that carry out an operation (e.g., executable code) and/or data. Executable code may be any executable code, e.g., an app/application, a program, a process, task or script.

The storage, such as storage 120 of system 100 of FIG. 1, may be, or may include, for example, one or more of a hard disk drive, a solid-state drive, an optical disc drive (such as DVD or Blu-Ray), a USB drive or other removable storage device, and/or other types of storage devices. Data such as instructions, code, procedure data, and health-related data, among other things, may be stored in the storage and may be loaded from storage into the memory where it may be processed by the processor or controller.

The illustrated components of FIG. 1 are exemplary and variations are contemplated to be within the scope of the present disclosure. For example, the numbers of components may be greater or fewer than as described and the types of components may be different than as described. When system 100 implements a machine learning system, for example, a large number of graphics processing units may be utilized. When system 100 implements a data storage system, a large number of storages may be utilized. As another example, when system 100 implements a server system, a large number of central processing units or cores may be utilized. Other variations and applications are contemplated to be within the scope of the present disclosure.

For purposes of explanation, specific configurations and details are set forth in order to provide a thorough understanding of aspects of the disclosed technology. However, it is apparent to one skilled in the art that the disclosed technology can be practiced without using every aspect presented herein.

Different aspects are disclosed herein. Features of certain aspects can be combined with features of other aspects; thus certain aspects can be combinations of features of multiple aspects.

While several embodiments of the disclosure have been described herein and/or shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A system for validated Electronic-Medical-Record (EMR) reporting, comprising:
at least one hardware processor; and
at least one computer readable storage device storing instructions for execution by the at least one hardware processor, the instructions, when executed, cause the system to:
access a set of measurements of one or more health indicators for one or more patients, the set of measurements comprising values of measurements of the set of measurements;
identify invalid values of measurements and abnormal values of measurements in the set of measurements;
indicate the identified invalid values of measurements and the identified abnormal values of measurements; and
report the set of measurements to an EMR system, wherein the reporting comprises:
for each measurement having an invalid value, reporting the measurement to the EMR system only after a valid value is received for the measurement and the invalid value is replaced by the valid value, and
for each measurement having an abnormal value, reporting the measurement to the EMR system only after the abnormal value is approved or a normal value is received for the measurement and the abnormal value is replaced by the normal value.

2. The system according to claim 1, wherein the instructions, when executed, further cause the system, for each measurement having an invalid value, to:
request a user to repeat the respective measurement, or
allow the user to edit the invalid value.

3. The system according to claim 2, wherein:
if the user has an adequate permission, the user is allowed to edit the invalid value, and
if the user does not have an adequate permission, the user is requested to repeat the respective measurement.

4. The system according to claim 1, wherein the instructions, when executed, further cause the system to allow the approval or the editing of an abnormal value of a measurment of the set of measurements only by a user having an adequate permission.

5. The system according to claim 1, wherein identifying invalid values of measurements in the set of measurements comprises determining a value of a measurement of the set of measurements as invalid if the value is not within a predefined range.

6. The system according to claim 1, wherein the identifying of the abnormal values of measurements in the set of measurements comprises evaluating each value of the values of the measurements of the set of measurements based on the medical condition of a patient from whom the respective measurement was taken.

7. The system according to claim 6, wherein:
each patient is associated with a predefined profile of a set of predefined profiles,
each profile of the set of predefined profiles comprises one or more ranges of values of one or more measurements of health indicators,
each range of values of the one or more ranges of values is defined for a different type of measurement, and
evaluating each value of the values of the measurements of the set of measurements comprises determining a value of a measurement of the set of measurements as abnormal if the value of the respective measurement is not within the range of values defined for the respective measurement in the predefined profile of the set of predefined profiles associated with the patient from whom the respective measurement was taken.

8. The system according to claim 1, wherein the reporting further comprises, for each measurement of the set of measurements missing a value and of a type defined as an essential type of measurement, reporting the respective measurement to the EMR system only after the lack of value is approved or a value is received for the respective measurement.

9. The system according to claim 8, wherein the instructions, when executed, further cause the system to receive the value for the respective measurement by allowing a user who has an adequate permission to enter the value.

10. The system according to claim 1, wherein the instructions, when executed, further cause the system to store in the storage device the values of the measurements of the set of measurements except for values which are invalid.

11. A computer-implemented method for validated Electronic-Medical-Record (EMR) reporting, comprising:
accessing a set of measurements of one or more health indicators for one or more patients, the set of measurments comprising values of measurements of the set of measurements;
identifying invalid values of measurements and abnormal values of measurements in the set of measurements;
indicating the identified invalid values of measurements and the identified abnormal values of measurements; and
reporting the set of measurements to an EMR system, wherein the reporting comprises:
for each measurement having an invalid value, reporting the measurement to the EMR system only after a valid value is received for the measurement and the invalid value is replaced by the valid value, and
for each measurement having an abnormal value, reporting the measurement to the EMR system only after the abnormal value is approved or a normal value is received for the measurement and the abnormal value is replaced by the normal value.

12. The computer-implemented method according to claim 11, further comprising requesting a user to repeat the respective measurement or allowing the user to edit the invalid value.

13. The computer-implemented method according to claim 12, wherein:
if the user has an adequate permission, the user is allowed to edit the invalid value, and
if the user does not have an adequate permission, the user is requested to repeat the respective measurement.

14. The computer-implemented method according to claim 11, further comprising allowing the approval or the editing of an abnormal value of a measurment of the set of measurments only by a user having an adequate permission.

15. The computer-implemented method according to claim 11, wherein identifying invalid values of measurements in the set of measurements comprises determining a value of a measurement of the set of measurements as invalid if the value is not within a predefined range.

16. The computer-implemented method according to claim 11, wherein the identifying of the abnormal values of measurements in the set of measurements comprises evaluating each value of the values of the measurements of the set of measurements based on the medical condition of a patient from whom the respective measurement was taken.

17. The computer-implemented method according to claim 16, wherein each patient is associated with a predefined profile of a set of predefined profiles, each profile of the set of predefined profiles comprises one or more ranges of values of one or more measurements of health indicators, and wherein each range of values of the one or more ranges of values is defined for a different type of measurement.

18. The computer-implemented method according to claim 17, wherein evaluating each value of the values of the measurements of the set of measurements comprises determining a value of a measurement of the set of measurements as abnormal if the value of the respective measurement is not within the range of values defined for the respective measurement in the predefined profile of the set of predefined profiles associated with the patient from whom the reapective measurement was taken.

19. The computer-implemented method according to claim 11, wherein the reporting further comprises, for each measurement of the set of measurements missing a value and of a type defined as an essential type of measurement, reporting the respective measurement to the EMR system only after the lack of value is approved or a value is received for the respective measurement.

20. The computer-implemented method according to claim 11, further comprising storing, in a storage device, the values of the measurements of the set of measurements except for values which are invalid.
